(19) United States
(12) Patent Application Publication
(10) Pub. No.: US 2009/0082839 A1
(43) Pub. Date: Mar. 26, 2009

(54) Electronic anti-snoring and sleep apnea device for sleep-breathing disorders, electronic anti-bruxing device, and electronic device for TMD therapy

(75) Inventors: Sherrill F. Lindquist, Melbourne, FL (US); John E. Zurasky, Merritt Island, FL (US); Jacob D. Zurasky, Merritt Island, FL (US)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(73) Assignee: ZURLIN TECHNOLOGIES HOLDINGS, LLC, Melbourne, FL (US)

(21) Appl. No.: 12/154,339

(22) Filed: May 22, 2008

Related U.S. Application Data
(60) Provisional application No. 60/946,159, filed on Jun. 26, 2007.

(51) Int. Cl. A61N 1/00

(52) U.S. Cl. 607/134

(57) ABSTRACT

Devices and methods for preventing or mitigating the occurrence of sleep breathing disorders use an extra-oral unit for detecting sounds from a sleeping patient and for sending commands to an intra-oral device for applying an electrical stimulation to one or more locations in the oral cavity of the patient. Similarly, bruxing and temporomandibular disorder can be mitigated or prevented by applying electrical stimulation to the oral cavity of a patient in response to the detection of pressure caused by bruxing activity or oral para-functional activity. The intra-oral unit includes a controller for controlling the generation of the electrical stimulation to be applied to the oral cavity of the patient, and first and second protective layers sealing the controller therebetween.

### CROSS-REFERENCE TO RELATED APPLICATIONS

The present invention claims priority from U.S. Provisional Application No. 60/946,159, filed Jun. 26, 2007, entitled “Electronic Anti-Snoring & Sleep Apnea Device (EAS/SAD) For Sleep-Breathing Disorders, Electronic Anti-Bruxing Device, And Electronic Device For TMD Therapy” by Lindquist et al., which is hereby incorporated by reference in its entirety.

### COPYRIGHT NOTICE

A portion of the disclosure of this patent document contains material which is subject to copyright protection. The copyright owner has no objection to the facsimile reproduction by any one of the patent document or the patent disclosure, as it appears in the Patent and Trademark Office patent file or records, but otherwise reserves all copyright rights whatsoever.

### REFERENCE TO PROGRAM LISTING

This patent application includes a computer program listing appendix submitted on a single compact disk, the contents of which are hereby incorporated by reference in their entirety. The compact disk, submitted in duplicate, includes source code for the nightstand unit and for the mouthpiece. These are identified as file Nightstand v1.7.asm, created on Apr. 14, 2008, file size 99 KB and Mouthpiece v1.3.asm, created on Apr. 14, 2008, file size 18 KB, respectively.

### BACKGROUND OF THE INVENTION

1. Field of the Invention

The invention is directed to systems for mitigating or eliminating sleep-disordered breathing, and, more particularly, to electronic devices for preventing snoring, sleep apnea, bruxing and for Temporomandibular Disorder (TMD) therapy.

2. Description of the Prior Art

Current treatments for snoring and Obstructive Sleep Apnea (OSA) include behavioral changes such as losing weight, avoiding alcohol, tobacco, sleeping pills, and attempting to adjust sleeping position. Continuous Positive Airway Pressure (CPAP) can be effective but very uncomfortable and noisy to wear during the night with only 50% patient compliance. Oral appliance therapy is available but many times can cause facial pain, TMD symptoms, and changes in tooth position and occlusion. Surgical approaches are available but most are quite drastic requiring patients to undergo unwanted procedures.

An example of one approach is presented in U.S. Pat. No. 5,792,067 to Karell which is directed to a device and method for addressing sleep and other disorders through electromuscular stimulation within specific areas of a patient's mouth. A mouthpiece includes an electrode for stimulating either the hard palate, soft palate or the pharynx. The mouthpiece includes a denture-like plate to which the control unit and electrodes may be attached.

### SUMMARY OF THE INVENTION

Objects, advantages and features in accordance with the present invention are provided by an electronic system for preventing or mitigating the occurrence of sleep breathing disorders, comprising: an intra-oral unit for placement in an oral cavity of a sleeping patient; and an extra-oral unit for detecting sounds from the sleeping patient and for sending at least one command to an intra-oral unit. The intra-oral unit is for applying, in response to the at least one command from the extra-oral unit, an electrical stimulation to at least one location in the oral cavity of the patient.

The intra-oral unit may comprise a wireless receiver for receiving commands from the extra-oral unit; and a controller associated with the wireless receiver and controlling the generation of the electrical stimulation to be applied to the oral cavity of the patient. The intra-oral unit may comprise a rechargeable battery to power the controller and/or a voltage boost circuit and waveform generation circuit driven by the controller to control a voltage, frequency and duration of the electrical stimulation applied to the oral cavity of the patient. The intra-oral unit may further comprise first and second plastic protective layers sealing the wireless receiver and controller therebetween, and/or include tissue contacts associated with the controller for applying the electrical stimulation to the oral cavity of the patient.

The extra-oral unit may include a microphone, a controller for receiving signals from the microphone and generating commands in response thereto, and a wireless transmitter associated with the controller for sending the commands to the intra-oral unit. The extra-oral unit may further comprise signal processing circuitry connected between the microphone and the controller to perform at least one of amplifying and filtering of the signals received by the controller. The controller may sample signals, and/or convert signals sampled from the microphone into digital signals, and store the samples, e.g. in a non-volatile memory associated with the controller. The extra-oral unit may further comprise a computing device link associated with the controller for outputting stored samples to an external device, and/or a user interface for displaying information from the controller and for inputting information to the controller.

A method aspect is for preventing or mitigating the occurrence of sleep breathing disorders comprising: detecting sounds from a sleeping patient; and applying an electrical stimulation to one or more locations in an oral cavity of the patient in response to detected sounds. Applying the electrical stimulation may include providing an intra-oral unit comprising: a wireless receiver for receiving commands; and a controller associated with the wireless receiver to control the generation of the electrical stimulation to be applied to the oral cavity of the patient. Applying the electrical stimulation may further comprise controlling a voltage, frequency and duration of the electrical stimulation applied to the oral cavity of the patient. Providing the intra-oral unit may further comprise sealing the wireless receiver and controller between first and second plastic protective layers, and/or applying the electrical stimulation to the oral cavity of the patient via tissue contacts associated with the controller.

Detecting sounds may comprise providing an extra-oral unit including: a microphone; a controller for receiving signals from the microphone and generating commands in response thereto; and a wireless transmitter associated with the controller for sending the commands to the intra-oral unit. Detecting sounds may further comprise at least one of amplifying and filtering of the signals received by the controller from the microphone and/or storing samples of the signals in a non-volatile memory associated with the controller of the extra-oral unit.

Objects, advantages and features in accordance with the present invention are provided by an electronic system for preventing or mitigating the occurrence of at least one of bruxing and temporomandibular disorder in a sleeping patient, comprising: an intra-oral unit, for placement in an oral cavity of the patient, and including at least one sensor to detect pressure from oral activity in the sleeping patient, a controller to control the generation of an electrical stimulation to be applied to the oral cavity of the patient in response to detected pressure above a threshold from the at least one sensor and at a level that will not awaken the patient, and tissue contacts associated with the controller for applying the electrical stimulation to the oral cavity of the patient. The intra-oral unit may further comprise a rechargeable battery to power the controller, and/or a voltage boost circuit and waveform generation circuit driven by the controller to control a voltage, frequency and duration of the electrical stimulation applied to the oral cavity of the patient. The intra-oral unit may further comprise first and second plastic protective layers sealing the controller therebetween.

A method aspect is for preventing or mitigating the occurrence of at least one of bruxing and temporomandibular disorder in a sleeping patient, the method comprising: detecting pressure from oral activity in a sleeping patient; and applying an electrical stimulation to an oral cavity of the patient in response to detected pressure above a threshold at a level that will not awaken the patient. Applying the electrical stimulation may comprise controlling a voltage, frequency and duration of the electrical stimulation applied to the oral cavity of the patient. Also, applying the electrical stimulation may comprise: providing an intra-oral unit, for placement in an oral cavity of the patient, and including at least one sensor to detect pressure from oral activity in the sleeping patient, a controller to control the generation of the electrical stimulation to be applied to the oral cavity of the patient in response to detected pressure, and tissue contacts associated with the controller for applying the electrical stimulation to the oral cavity of the patient. Providing the intra-oral unit may further comprise sealing the controller between first and second plastic protective layers.

The present invention provides effective treatment for snoring and OSA in mild to moderate cases including snoring and upper airway resistance syndrome by opening the patient's airway by flexing or stiffening the soft palate (levator veli palatine and tensor veli palatine) along with the uvula through the delivery of a very mild electric current introduced by a battery operated, electronic receiver located within the palatal aspect of the intra-oral appliance.

This is achieved through the use of an electronic system which employs a custom fabricated intra-oral appliance which stimulates certain intra-oral tissues, and an extra-oral unit which receives patient snoring sounds within a certain frequency and then transmits a signal to the intra-oral appliance initiating the necessary intra-oral stimulation to aid in opening the patient's partially or totally collapsed airway. The invention permits ready treatment of snoring and sleep apnea, bruxism and TMD.

One aspect of the invention is directed to an electronic system for preventing or mitigating the occurrence of sleep breathing disorders. An extra-oral unit is used for detecting sounds from a sleeping patient and for sending at least one command to an intra-oral unit, which in response to a command from the extra-oral unit applies an electrical stimulation to one or more locations in the oral cavity of said patient.

Another aspect of the invention is directed to a technique for preventing or mitigating the occurrence of bruxing and TMD by detecting pressure from bruxing activity or parafunctional activity in a sleeping patient, and applying an electrical stimulation to one or more locations in the oral cavity of said patient in response to detected pressure above a threshold.

### BRIEF DESCRIPTION OF THE DRAWINGS

FIG. 1 is a drawing of a maxillary stone cast with a thin plastic sheet adapted to it used to fabricate the maxillary plastic arch form for the electronic components of the intra-oral appliance in accordance with the present invention.

FIG. 2 is a drawing of the rechargeable battery and electronic transceiver located in the palatal aspect of the intra-oral appliance. Also displayed are the circuit extension leads and contacts which stimulate the hamular notches.

FIG. 3A is a bottom view of the intra-oral appliance including the electronics being sandwiched between thin protective layers.

FIG. 3B is a cross-sectional view of the intra-oral appliance taken along the line B-B of FIG. 3A.

FIG. 4 is a drawing of the extra-oral unit housing the microphone, signal processor, battery charger, and the data recorder which is placed on the patient's nightstand.

FIGS. 5A and 5B are simplified charts of the electronic functions of a first version of the remote unit and intra-oral appliance, respectively.

FIG. 6 is a drawing of the intra-oral appliance for bruxism showing bruxism detection sensors in the form of a pressure sensitive electro conductive rubber sensor or pressure receptor switch and the electrical stimulation points.

FIG. 7 is a drawing of the intra-oral appliance for TMD showing design with pressure sensitive electro conductive rubber sensors or pressure receptor switches to detect occlusal para-function and the electrical stimulation points.

FIG. 8 is a high-level block diagram of the hardware architecture of a mouthpiece unit in accordance with one aspect of the invention.

FIG. 9 is a high-level block diagram of the hardware architecture of a nightstand unit in accordance with one aspect of the invention.

FIGS. 10-12 are more detailed schematic diagrams of the hardware architecture of the mouthpiece unit and nightstand unit of FIGS. 8 and 9.

FIG. 13 is a high-level block diagram of the software architecture implemented in firmware for the mouthpiece unit in accordance with one aspect of the invention.

FIG. 14 is a high-level block diagram of the software architecture implemented in firmware for the nightstand unit in accordance with one aspect of the invention.

### DETAILED DESCRIPTION OF THE INVENTION

The present invention will now be described more fully hereinafter with reference to the accompanying drawings in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout. The dimensions of layers and regions may be exaggerated in the figures for greater clarity.

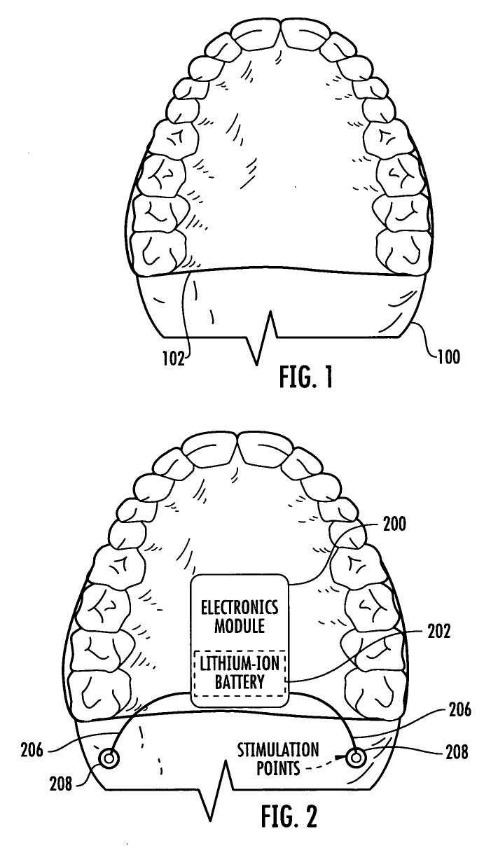

FIG. 1 is an illustration of a snoring and OSA patient's maxillary teeth. The cast 100 is fabricated by the dentist or dental assistant making alginate (irreversible hydrocolloid) impressions of the maxillary and mandibular arches in the usual way impressions are made. A vacuum thermoforming machine (such as manufactured by Raintree Essix Inc., Metairie, La.) can be used to pull down sufficiently heated plastic onto the maxillary model, as would be appreciated by those skilled in the art. This plastic material 102 will become the arch form base upon which a rechargeable battery and the electronic transceiving unit will be mounted. After these components are mounted in the palatal aspect of the arch, a second “sandwiching” piece of thin plastic is vacuum formed over the electronic components to protect them from saliva.

FIG. 2 is an illustration of the electronics module or transceiving unit 200 including rechargeable battery 202, and circuit extension leads 206 and associated tissues contacts 208 which contact the hammular notches bilaterally. The battery 202 used in the unit must be of sufficient voltage in order to create the necessary tone in the musculature involved with soft palate flexing or stiffening (tensor veli palatini muscles and the levator veli palatini muscles). When not in use, the intra-oral member should be recharged during the day. Wire leads 206 from the electronic circuit are preferably 28 gauge wire and run between the “sandwiched” plastic arch form distal to the maxillary 2nd molars and terminate with the circuit extension contacts 208, such as stainless orthodontic ballclamps (0.28 in (0.7 mm)) which contact in the hamular notch.

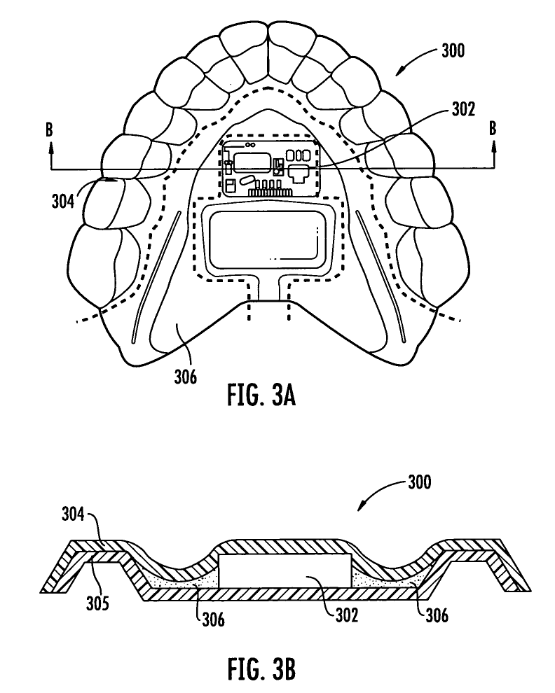

An example of the intra-oral appliance or mouthpiece 300 is illustrated in FIGS. 3A and 3B. The electronics module 302 is sandwiched between upper and lower protective layers 304, 305 (e.g. such as thermoformed plastic layers) for protection of the circuitry from saliva and associated corrosion. Also, an adhesive layer 306 (e.g. a bonded, light-cured, acrylic gel, such as Triad Gel from the Dentsply International of York, Pa.) is preferably applied between the protective layers, e.g. at a periphery thereof, to further aid in the corrosion prevention.

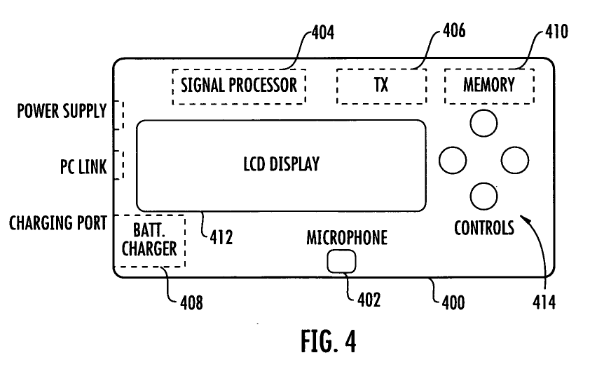

FIG. 4 is an illustration of the extra-oral electronic transceiving (nightstand) unit 400 which may be located on the patient's nightstand. It contains the microphone 402, a signal processor 404, and a wireless transceiver 406 to activate the intra-oral appliance. It also includes a battery charger 408 for the appliance and a data recorder 410 to monitor snoring/gasping frequency throughout the night. The location of the LCD display 412, microphone 402 and the controls 414 may be located as desired anywhere on the housing of the nightstand unit 400. In use, these components can be located not only on a nightstand but also anywhere proximate to the patient that may be desired.

The battery charger 408 of the extra-oral unit 400 and the associated battery 302 of the intra-oral unit may utilize connectors manufactured by 3M such as 0.100″ pin strip headers and 0.100″ board mount sockets. The socket is used in the mouthpiece and is sealed within the protective thin plastic layers by applying bonded, light-cured, acrylic gel, such as Triad Gel from the Dentsply International of York, Pa., to prevent moisture from entering the mouthpiece. Contactless charging, such as electromagnetic, capacitive and/or inductive charging may also be provided instead of the connectors.

To detect a snoring pattern, a computing element such as a microcontroller, monitors incoming audio signals from the microphone. When this becomes greater than or equal to the user-set threshold, electrical stimulation occurs. The active low pass filter attenuates sounds greater than 1 khz. Previous studies have identified a narrow band in which the majority of snoring sounds occur and with selective amplification of the input, bed partner and background noise will not reach threshold. The microphone input is relative to the distance from the noise source. Distance from the microphone on the nightstand next to the snorer and adjustability of sensitivity will prevent accidental activation.

Snoring does occur in variable patterns that will be recorded relative to timing and amplitude. The LCD screen shown in FIG. 4 has a line for displaying the number of snores over an eight-hour period and downloading the stored data to a computer program will produce a graph showing when the snores occurred, the number, and loudness. The patient will be able to evaluate their snoring with the intra-oral appliance in or out of the mouth and can set a time delay upon retiring before activation of the appliance. Evaluation of the recorded data will guide the adjustments to maximize the benefit for individual differences.

A PC link allows data transfer for home computer analysis and tracking of abnormal breathing sounds with and without the appliance in place. This will give the patient feedback on breathing difficulties during sleep and benefit of the appliance. The device functions by the extra-oral electronic unit detecting snoring sounds and, consequently, transmitting a wireless signal to the intra-oral appliance which, in turn, generates a low voltage current which is carried to the patient's hamular notches causing the soft palate to flex or stiffen aiding in the opening of the airway and restoring air flow to the patient's lungs.

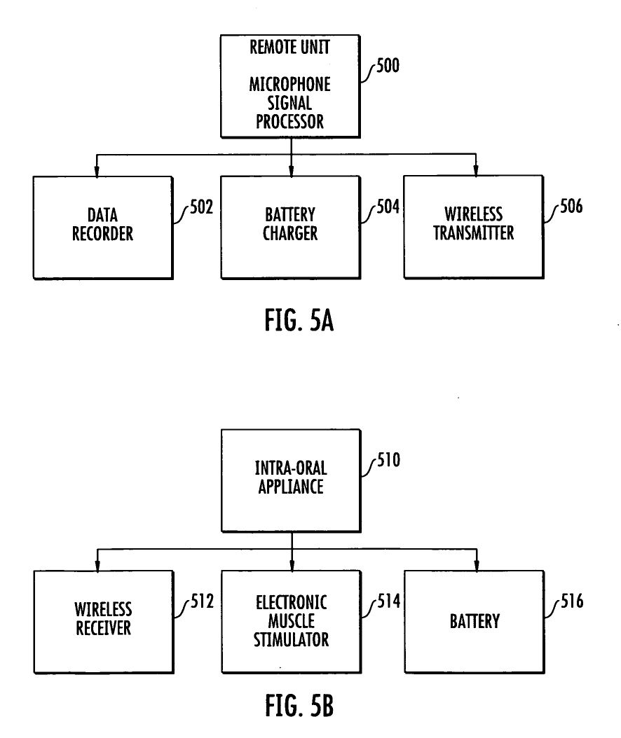

FIGS. 5A and 5B show a simplified chart of the electronic functions of a first version of the remote unit and intra-oral appliance, respectively. More specifically, referring to FIG. 5A, the extra-oral unit or remote unit operations include the microphone function and signal processing 500 which are associated with data recording 502, battery charging 504 and wireless transmissions 506. Referring now to FIG. 5B, the intra-oral appliance operations 510 are associated with wireless reception 512, electronic muscle stimulation 514 and power supply 516 from the battery.

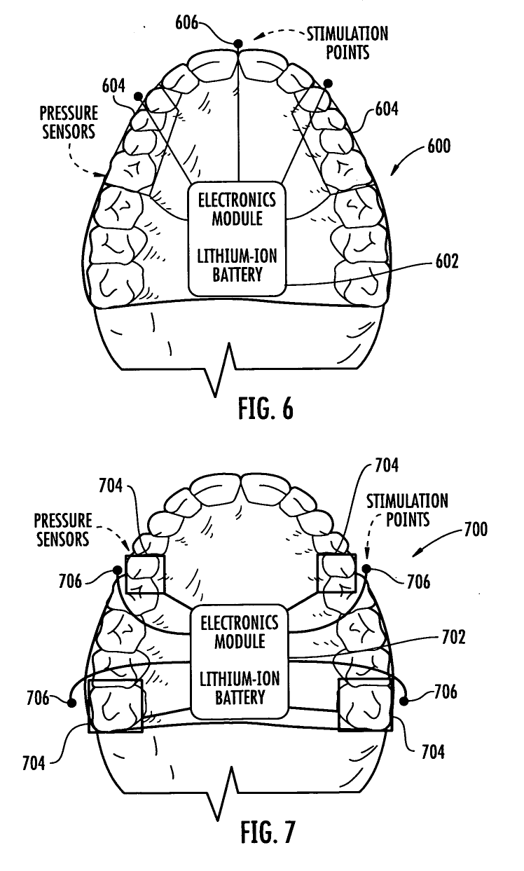

FIG. 6 is an illustration of the intra-oral appliance 600 for bruxism. This electronic orthosis works as a gnathologic appliance to protect teeth from damage during excursive movements. In addition, the electronics package 602 detects bruxing activity using a pressure electro conductive rubber sensor or pressure receptor switch 604 such as made by Bridgestone in Tokyo, Japan and stops it with electronic stimulation, via tissue contact 606, to the intra-oral mucosa at a subconscious level without sleep interruption. Patient adjustability and monitoring is available with the extra-oral unit, discussed above, that is in wireless communication with the intra-oral appliance.

FIG. 7 is an illustration of the intra-oral appliance 700 for TMD. Temporomandibular disorder (TMD), or TMJ syndrome, is a term covering acute or chronic inflammation of the temporomandibular joint, which connects the lower jaw to the skull. This orthotic type appliance detects oral para-functional activity through the use of pressure sensors 704 and an electronics package 702 in the appliance. A para-functional habit or parafunctional habit is the habitual exercise of a body part in a way that is other than the most common use of that body part. The term is most commonly used by dentists, orthodontists, or maxillofacial specialists to refer to parafunctional uses of the mouth, tongue and jaw. Oral para-functional habits may include bruxism (tooth-clenching or grinding), tongue tension, mouth-breathing, and any other habitual use of the mouth unrelated to eating, drinking, or speaking. Treatment includes electronic stimulation, via tissue contact 706 in response to detected pressure.

Wireless communication with the extra-oral unit provides data storage and patient adjustability for electrical stimulation in voltage, frequency, pulse width, and duration.

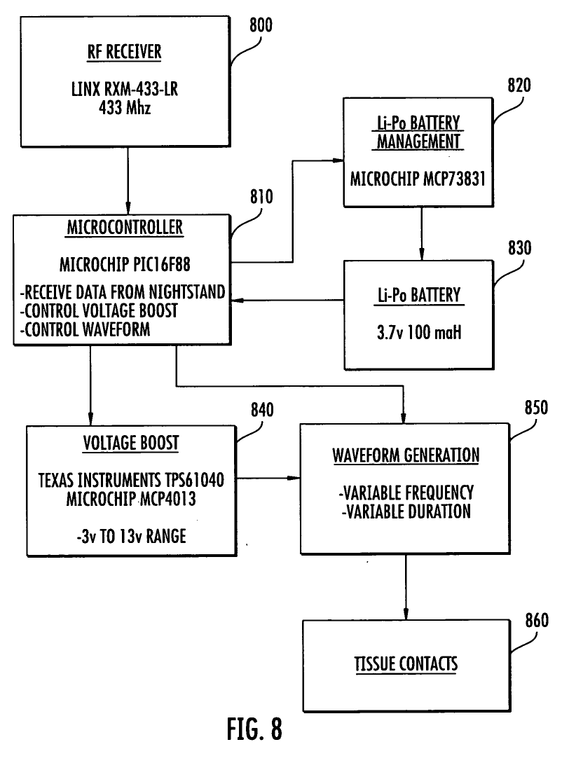

FIG. 8 is a high-level block diagram of a preferred hardware architecture of the mouthpiece unit in accordance with one aspect of the invention. An RF receiver 800, such as receiver RXM-433-LR manufactured by Linx Technologies, Inc. of Merlin, Oreg., receives signals transmitted by the nightstand unit, described hereinafter. Signals from the RF receiver are passed to a computing element such as controller or microcontroller 810 which is preferably a PIC16F88 microcontroller manufactured by Microchip Technology Inc. of Chandler, Ariz. The Voltage Boost 840 receives the output of the RF receiver and provides a voltage boost, preferably using switch boost converter TPS61040 manufactured by Texas Instruments, Inc. of Dallas, Tex., to boost the voltage as specified by the microcontroller. The microcontroller also controls the shape of the waveform generator 850 to vary the frequency and duration of the waveforms applied to the mouth of the patient through tissue contacts 860. Control of the intensity of the waveform can be exerted using an MCP4013 Digital Potentiometer manufactured by Microchip Technology, Inc. of Chandler, Ariz.

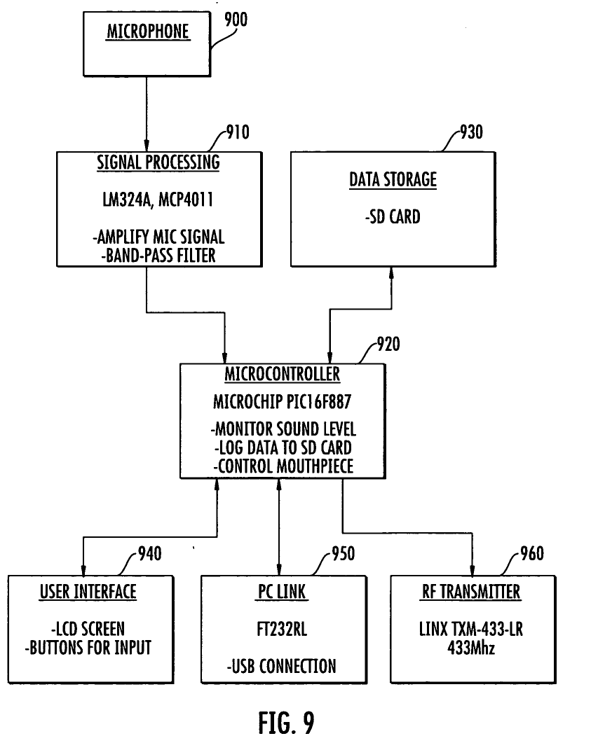

FIG. 9 is a high-level block diagram of a preferred hardware architecture of the nightstand unit in accordance with one aspect of the invention. The so-called nightstand unit includes a microphone 900, the purpose of which is to detect sounds that occur during sleep. For purposes of this application it is called a nightstand unit although the particular unit or its components can be located anywhere in the vicinity of the person who might be the subject of a sleep-disordered breathing. Sounds picked up by the microphone 900 during operation of the unit, usually at night, is passed to a signal processing unit 910. The purpose of the signal processing unit 910 is to amplify the signal from the microphone and shifts it into the 0 to 5 volt range, preferably. This is preferably done using a quad operational amplifier LM324A manufactured by STMicroelectronics of Phoenix, Ariz. The processed signal from 910 is passed to a computing element such as the controller or microcontroller 920 which is preferably a microcontroller PIC16F887 manufactured by Microchip Technology, Inc. of Chandler, Ariz. The amplified signal is sampled by microcontroller 920 and the sample stored in a data storage unit 930 which is preferably a standard SD memory card where it will be stored. The microcontroller 920 is programmed, as described more hereinafter and in the source code CD provided with this application, to monitor the sound level in the room. When the level indicates that a certain sleep-breathing disorder is present, such as snoring, it sends a signal to the RF transmitter 960 to activate the mouthpiece unit, previously described. This results in electrical stimulation of the oral cavity of the patient at the tissue contacts 860 shown in FIG. 8. The electrical stimulation is set so as not to interrupt the sleep of the patient but rather to stimulate the oral cavity to aid in opening the patient's partially or totally collapsed airway. The nightstand unit also includes a link to a personal computer 950 which may be either a wired connection or a wireless connection over which data from the data storage unit 930 can be downloaded and analyzed. Access to the microcontroller is also provided over user interface 940 which displays information from the microcontroller and allows the user to activate buttons or controls to indicator set various preferences with respect to the operation of the unit.

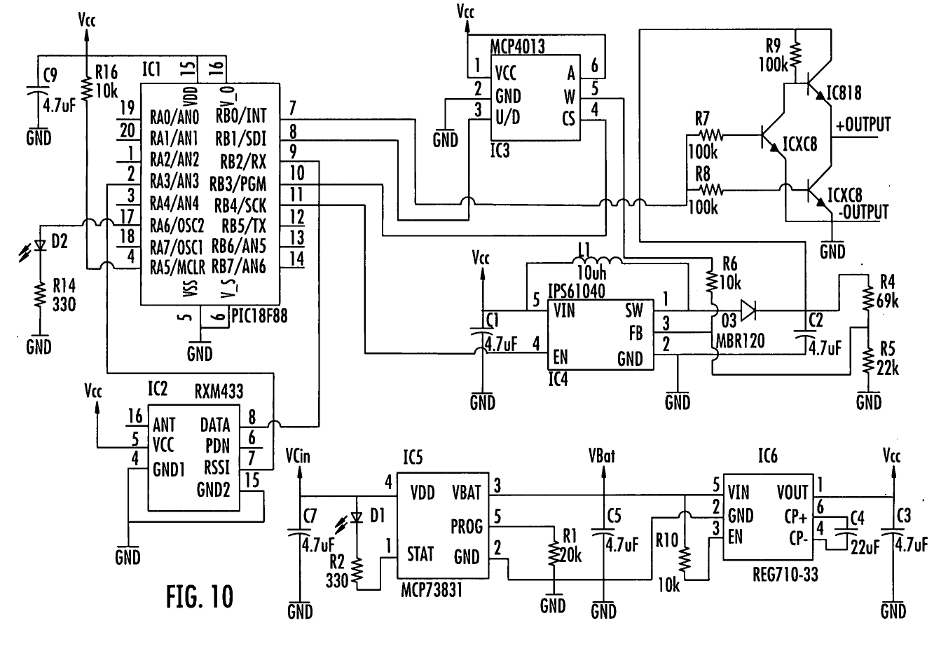

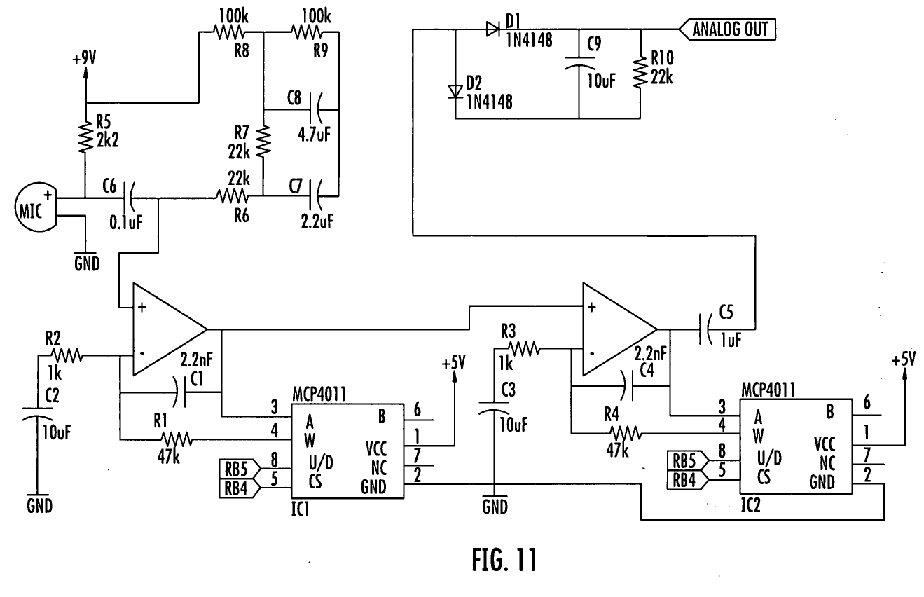

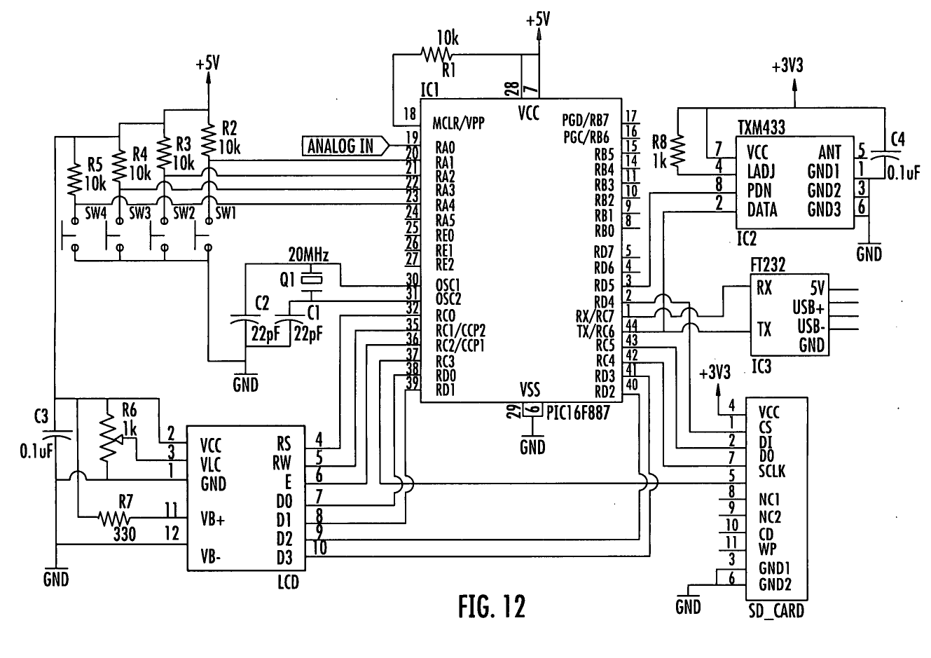

Referring to FIGS. 10-12, more detailed schematic diagrams of an embodiment of the mouthpiece unit and nightstand unit are illustrated. More specifically, FIG. 10 illustrates the various integrated circuit chips and connections of an embodiment of the mouthpiece of FIG. 8 including the microcontroller, RF receiver, battery and associated battery management, voltage boost, waveform generation and tissue contacts as shown. FIG. 11 illustrates the various integrated circuit chips and connections of an embodiment of the microphone and signal processing circuitry of the nightstand unit of FIG. 9. FIG. 12 illustrates the various integrated circuit chips and connections of an embodiment of the microcontroller, data storage, user interface, PC link and RF transmitter of the nightstand unit of FIG. 9.

Exchange of information between the mouthpiece and the nightstand unit occurs in data packets. A single (nightstand) unit can service up to 256 mouthpiece units on separate channels.

The format of the data packet being sent to the mouthpiece consists of 11 bytes of data and is as follows:

Three start bytes: 0x01, 0x03, 0x54

Channel byte: 0x00 to 0xFF

Command byte: 0x01 to 0x04

Argument byte 1: 0x00 to 0xFF

Argument byte 2: 0x00 to 0xFF

Argument byte 3: 0x00 to 0xFF

Three end bytes: 0x07, 0x0E, 0x56

The mouthpiece knows what channel it is on and will not respond to any data packets that are not addressed to its specific channel. The only command that supersedes channel designations is command 0x03, which is the command for the mouthpiece to display what channel it is currently set to (by flashing the display LED).

Command overview:

0x01—Stimulation Command

Argument 1—Intensity Setting

Argument 2—Frequency Setting

Argument 3—Duration Setting

0x02—Change Mouthpiece Channel

Argument 1—New Channel

Argument 2—Null

Argument 3—Null

0x03—Display Current Channel

Argument 1—Null

Argument 2—Null

Argument 3—Null

0x04—Toggle Display Light On/Off

Argument 1—Null

Argument 2—Null

Argument 3—Null

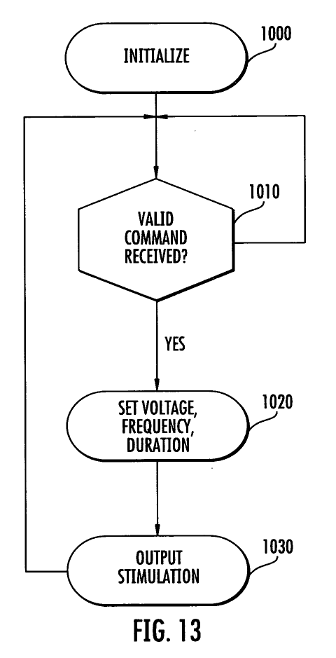

FIG. 13 is a high-level block diagram of the software architecture implemented in firmware for the mouthpiece unit in accordance with one aspect of the invention. Complete source code for this software is set forth in the attached CD ROM appendix and is contained in the file labeled Mouthpiece v1.3.asm, created on Apr. 14, 2008, file size 18 KB.

At a high-level, the firmware for the mouthpiece has an initialization state 1000 which readies the mouthpiece unit to receive signals from the nightstand unit. If the mouthpiece unit receives a valid command or signal from the nightstand unit (1010) the voltage, frequency and duration is set (1020) and the output stimulation, corresponding to the setting, is applied to the patients oral cavity. Once stimulated, the mouthpiece software waits until another command is received. This process loops throughout the night, until the device is turned off when the patient awakes in the morning.

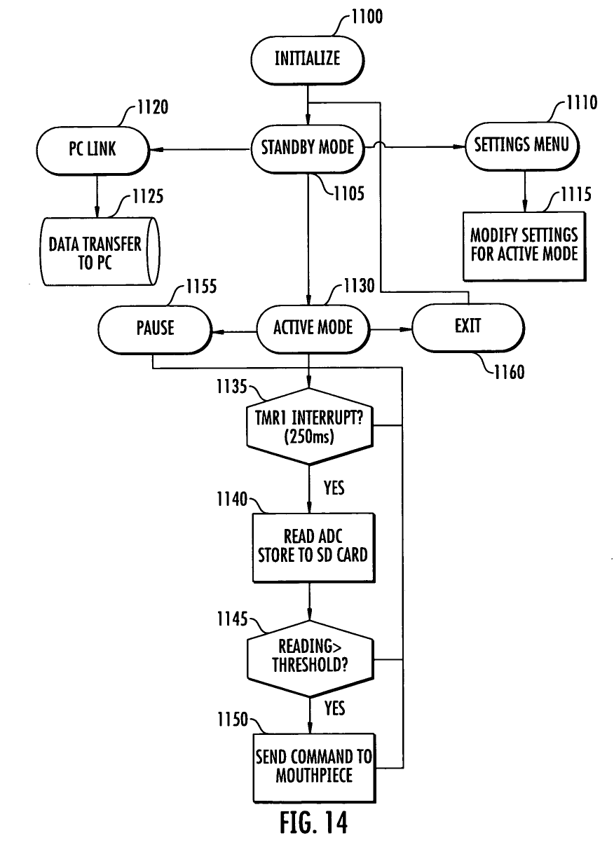

FIG. 14 is a high-level block diagram of the software architecture implemented in firmware for the nightstand unit in accordance with one aspect of the invention. Again, complete source code for the software represented in this figure is contained in the CD ROM Appendix and is labeled with the file name Nightstand v1.7.asm, created on Apr. 14, 2008, file size 99 KB.

When turned on, the nightstand software initializes (1100) the nightstand unit for operation.

The software then enters a standby mode 1105. In the standby mode, the nightstand unit can receive settings set by a user through a settings menu 1110. The settings also permit the software to be transitioned into active mode (1115). From the standby mode 1105, the software can also enter into a linking operation with a computing device, such as a personal computer over PC link 1120. When in communication with the PC over PC link 1120, the nightstand unit can transfer data to a computing device where it can be stored and analyzed (1125). In active mode, the software enters a sampling loop during which the level of signal received from the microphone is sampled by asserting a timed interrupt, preferably every 250 milliseconds. The sampled signal will be converted to digital using an analog-to-digital function and the results stored in a storage unit, such as an SD card (1140) for later analysis. If the sampled value is above a threshold (1145) a command is sent to the mouthpiece (1150) where it is received and, as previously discussed, will be utilized to initiate electrical stimulation of the patient's oral cavity. This timer driven interrupt sequence occurs repeatedly throughout the night but may be paused (1155) or exited (1160) upon user action.

This new appliance detects and records specific snoring frequencies with a nightstand unit that selectively activates a wireless gnathodynamics based electronic intra-oral appliance to stop the snore. A low voltage electrical stimulation of the levator and tensor palatine muscles stops the snore. The resulting increase in muscle tonicity restores the airway and prevents vibration of the soft palate without awaking the patient. It is prescribed by the dentist and fabricated by a certified dental laboratory using pre-packaged electronic circuitry and a rechargeable battery that is encapsulated between two layers of thermoformed material. The mandible is positioned anatomically considering the temporomandibular joints, muscles, and teeth. All teeth are in contact to prevent extrusion and all eccentric movements are sheltered with a mutually protected occlusal scheme built into the appliance with no anterior repositioning or excessive mandibular opening. Overnight data is recorded preferably every 250 msec and stored for download to any PC. Analysis of stored data by the dentist preferably guides adjustments for muscle stimulation relative to intensity, duration, frequency, sensitivity, and time delay.

Electronic muscle stimulation restores tone while sleeping to that experienced during the day. The increased tonicity prevents the soft palate from vibrating on inspiration and expiration. In initial clinical trials to determine that the invention works, the results with four chronic snoring patients showed effectiveness, patient acceptance, and ease of use, have been exceptional. A statistically significant decrease in snoring sound levels were recorded. Witnesses confirmed decreased snoring activity and patients stated that they felt more rested and were having dreams (REM sleep) again. Pulse oximetry data shows increased average oxygen saturation levels with appliance use. No occlusal changes, patient discomfort, or TMD symptoms were noted after four months of wear.

While various embodiments of the present invention have been illustrated herein in detail, it should be apparent that modifications and adaptations to those embodiments may occur to those skilled in the art without departing from the scope of the present invention as set forth in the following claims.

What is claimed is:

1. An electronic system for preventing or mitigating the occurrence of sleep breathing disorders, comprising:
an intra-oral unit for placement in an oral cavity of a sleeping patient; and
an extra-oral unit for detecting sounds from the sleeping patient and for sending at least one command to an intra-oral unit;
the intra-oral unit comprising a wireless receiver for receiving commands from the extra-oral unit, a controller associated with the wireless receiver and controlling the generation of the electrical stimulation to be applied to the oral cavity of the patient, and first and second protective layers sealing the wireless receiver and controller therebetween;
the intra-oral unit for applying, in response to the at least one command from the extra-oral unit, an electrical stimulation to at least one location in the oral cavity of the patient.

2. The electronic system of claim 1 wherein the intra-oral unit further comprises an adhesive layer between the first and second protective layers and surrounding the wireless receiver and controller.

3. The electronic system of claim 1 wherein the intra-oral unit comprises a rechargeable battery to power the controller.

4. The electronic system of claim 1 wherein the intra-oral unit comprises a voltage boost circuit and waveform generation circuit driven by the controller to control a voltage, frequency and duration of the electrical stimulation applied to the oral cavity of the patient.

5. The electronic system of claim 1 wherein the first and second protective layers comprise first and second thermoformed plastic layers sealing the wireless receiver and controller therebetween.

6. The electronic system of claim 1 wherein the intra-oral unit further comprises tissue contacts associated with the controller for applying the electrical stimulation to the oral cavity of the patient.

7. The electronic system of claim 1 wherein the extra-oral unit comprises:
a microphone;
a controller for receiving signals from the microphone and generating commands in response thereto; and
a wireless transmitter associated with the controller for sending the commands to the intra-oral unit.

8. The electronic system of claim 7 wherein the extra-oral unit further comprises signal processing circuitry connected between the microphone and the controller to perform at least one of amplifying and filtering of the signals received by the controller.

9. The electronic system of claim 7 wherein the controller samples signals from the microphone and stores the samples.

10. The electronic system of claim 9 wherein the controller converts signals sampled from the microphone into digital signals.

11. The electronic system of claim 9 wherein the extra-oral unit further comprises a non-volatile memory associated with the controller and for storing the samples therein.

12. The electronic system of claim 9 wherein the extra-oral unit further comprises a computing device link associated with the controller for outputting stored samples to an external device.

13. The electronic system of claim 7 wherein the extra-oral unit further comprises a user interface for displaying information from the controller and for inputting information to the controller.

14. A method for preventing or mitigating the occurrence of sleep breathing disorders comprising:
detecting sounds from a sleeping patient; and
applying an electrical stimulation to one or more locations in an oral cavity of the patient in response to detected sounds, including providing an intra-oral unit comprising
a wireless receiver for receiving commands,
a controller associated with the wireless receiver to control the generation of the electrical stimulation to be applied to the oral cavity of the patient, and
first and second protective layers sealing the wireless receiver and controller therebetween.

15. The method of claim 14 wherein providing the intra-oral unit further comprises applying an adhesive layer between the first and second protective layers and surrounding the wireless receiver and controller.

16. The method of claim 15 wherein applying the electrical stimulation further comprises controlling a voltage, frequency and duration of the electrical stimulation applied to the oral cavity of the patient.

17. The method of claim 15 wherein the first and second protective layers comprise first and second thermoformed plastic layers sealing the wireless receiver and controller therebetween.

18. The method of claim 15 wherein providing the intra-oral unit further comprises applying the electrical stimulation to the oral cavity of the patient via tissue contacts associated with the controller.

19. The method of claim 15 wherein detecting sounds comprises providing an extra-oral unit including:
a microphone;
a controller for receiving signals from the microphone and generating commands in response thereto; and
a wireless transmitter associated with the controller for sending the commands to the intra-oral unit.

20. The method of claim 19 wherein detecting sounds further comprises at least one of amplifying and filtering of the signals received by the controller from the microphone.

21. The method of claim 19 wherein detecting sounds further comprises storing samples of the signals in a non-volatile memory associated with the controller of the extra-oral unit.

22. An electronic system for preventing or mitigating the occurrence of at least one of bruxing and temporomandibular disorder in a sleeping patient, comprising:
an intra-oral unit, for placement in an oral cavity of the patient, and including
at least one sensor to detect pressure from oral activity in the sleeping patient,
a controller to control the generation of an electrical stimulation to be applied to the oral cavity of the patient in response to detected pressure above a threshold from the at least one sensor and at a level that will not awaken the patient,
first and second protective layers sealing the at least one sensor and controller therebetween, and
tissue contacts associated with the controller for applying the electrical stimulation to the oral cavity of the patient.

23. The electronic system of claim 22 wherein the intra-oral unit further comprises a rechargeable battery to power the controller.

24. The electronic system of claim 22 wherein the intra-oral unit further comprises a voltage boost circuit and waveform generation circuit driven by the controller to control a voltage, frequency and duration of the electrical stimulation applied to the oral cavity of the patient.

25. The electronic system of claim 22 wherein the first and second protective layers comprise first and second thermoformed plastic layers sealing the at least one sensor and controller therebetween.

26. A method for preventing or mitigating the occurrence of at least one of bruxing and temporomandibular disorder in a sleeping patient, the method comprising:
detecting pressure from oral activity in a sleeping patient; and
applying an electrical stimulation to an oral cavity of the patient in response to detected pressure above a threshold at a level that will not awaken the patient, including providing an intra-oral unit, for placement in an oral cavity of the patient, and comprising
at least one sensor to detect pressure from oral activity in the sleeping patient,
a controller to control the generation of the electrical stimulation to be applied to the oral cavity of the patient in response to detected pressure,
first and second protective layers sealing the at least one sensor and controller therebetween, and
tissue contacts associated with the controller for applying the electrical stimulation to the oral cavity of the patient.

27. The method of claim 26 wherein applying the electrical stimulation comprises controlling a voltage, frequency and duration of the electrical stimulation applied to the oral cavity of the patient.

28. The method of claim 26 wherein the first and second protective layers comprise first and second thermoformed plastic layers sealing the at least one sensor and controller therebetween.

29. The method of claim 28 wherein providing the intra-oral unit further comprises applying an adhesive layer between the first and second plastic protective layers and at least surrounding the controller.
